(19) United States
(12) Patent Application Publication
(10) Pub. No.: US 2003/0013767 A1
(43) Pub. Date: Jan. 16, 2003

(54) Method of treating weight loss using creatine

(76) Inventor: Samuel Bessman, Los Angeles, CA (US)

Correspondence Address: ROBERT BERLINER, FULBRIGHT & JAWORSKI LLP, 865 S. Figueroa St. #2900, Los Angeles, CA, 90017, US

(21) Appl. No.: 10/193,853

(22) Filed: Jul. 11, 2002

Related U.S. Application Data
(60) Provisional application No. 60/305,489, filed on Jul. 13, 2001.

(51) Int. Cl. A61K 31/198

(52) U.S. Cl. 514/565

(57) ABSTRACT

A method of using a creatine compound to treat muscle loss associated with liver and kidney diseases. In preferred embodiments, creatine monohydrate is administered by dialysis. The method can be extended to other diseases or conditions associated with muscle loss. Also provided is a composition comprising a dialysis fluid containing a creatine compound.

### CROSS-REFERENCE TO RELATED APPLICATIONS

This application is based on provisional application No. 60/305,489, filed on Jul. 13, 2001

### FIELD OF THE INVENTION

This invention relates generally to a treatment for muscle loss, and in particular to the use of creatine to counteract muscle loss associated with liver and kidney diseases.

### BACKGROUND OF THE INVENTION

Creatine is synthesized by the normal body and employed primarily in heart and skeletal muscle for growth and function. The normal human adult body contains about 100 grams of creatine, 95% of which is found in these two organs. About two-thirds of the creatine in muscle and heart is combined with inorganic phosphate to form creatine phosphate. In this form, creatine phosphate contains energy utilizable for contraction or synthesis of protein equal to the well known adenosine triphosphate (“ATP”), which is used for almost all energy requiring reactions of the organism. In contrast to ATP, creatine phosphate is used primarily for motion and growth of skeletal muscle.

Three natural amino acids, glycine, arginine and methionine, all of which are non-essential amino acids which can be synthesized by the normal body, provide the building blocks for creatine biosynthesis. About 2 grams of creatine are made every day. The first step of synthesis occurs primarily in the kidney, the second step in the liver. First, a guanidine group is transferred from arginine to glycine to form guanidoacetic acid, which enters the general circulation through the renal veins. Second, in liver cells, guanidoacetic acid receives a methyl group from methionine to become methylguanidoacetic acid, or creatine. This second step is catalyzed by the enzyme guanidoacetic methyltransferase. The creatine thus synthesized is carried by the blood to skeletal muscle, the heart, and in small amounts to the brain. It is noteworthy that creatine is not used in anyway by the organs in which it is made, nor is it made in the organs in which it is used, nor is it consumed during the performance of its function.

Creatine is a component of all skeletal muscle and as such is found in dietary meat. Very careful balance studies on the formation and excretion of creatine take this into consideration. A small, as yet unknown amount of creatine is made in the pancreas, but this amount is less than 2% of the creatine synthesized in the kidney-liver process. About 2% of the total body creatine is found in the brain, but no evidence has been adduced that creatine is made in that organ. In the case of vegetarians, there is no dietary contribution to the daily availability of creatine, but there does not seem to be any difficulty in obtaining sufficient creatine by endogenous synthesis. The present inventor is not aware of any studies of creatine availability in vegetarian weight loss situations. It may be found medically useful to add creatine to a vegetarian's diet to overcome weight loss.

Creatine has been administered for treatment of gyrate atrophy, based on the normal presence of creatine in the eye at levels of about one percent those found in muscle. There has been no effect on the atrophy, but there has been a slight strengthening of the periocular muscles. Two clinical syndromes involving severe deficiency of creatine in brain have been described in children. There is more or less severe associated mental retardation. The children have been treated with some success with oral creatine for periods of ten to twelve months at dosages of 4 grams/day (305-370 mg of creatine/kg body weight/day) to 8 grams/day (600-740 mg/kg/day) with no report of toxicity (6,7). Also, creatine has been tried therapeutically in animal models of neurologic diseases, such as amyotrophic lateral sclerosis, without success.

Creatine is best known for its role in the regeneration of ATP consumed during muscle contraction. Biochemical studies, including those carried out by the inventor and his colleagues (1), provide evidence for a creatine phosphate shuttle, a concept first reported by the inventor in 1972 (2). According to this concept, mitochondria phosphorylate creatine to produce creatine phosphate, which then diffuses to muscle myofibrils to act in the re-synthesis of ATP. Rather than behaving merely as an energy buffer, creatine plays a key role in energy metabolism of muscle by transporting energy from the mitochondria to the myofibril.

Other studies by the inventor and his colleagues provide evidence for an important role of creatine in muscle cell protein synthesis. In one study, a preferential inhibitor of creatine phosphokinase—the enzyme that reversibly catalyzes the addition of phosphate from ATP to creatine to form ADP and creatine phosphate—was found to inhibit protein synthesis in muscle cells at far lower tissue concentrations than in liver cells, which have very little creatine phosphokinase (3). One explanation for the enhanced sensitivity of muscle cells to creatine phosphokinase inhibition is that protein synthesis in muscle cells cannot proceed in the absence of creatine phosphate. In another study, in vitro protein synthesis in isolated polysomes from rat skeletal muscle was found to be much greater with added creatine phosphate than with ATP alone (4). The results of these studies support the view that creatine phosphate has a direct stimulatory effect on protein synthesis in muscle. Studies by others support this view of creatine phosphate action (5).

Severe weight loss regularly accompanies liver and kidney diseases. This loss is primarily due to significant wastage of skeletal muscle. In patients awaiting a liver or kidney transplant, weight loss contributes to the patients' frequent failure to survive until the transplant takes place. Even milder degrees of liver or kidney failure can be associated with weight loss.

Loss of weight in renal and liver patients, with attendant skeletal muscle wastage, is a metabolic consequence of the body's basic function of supplying glucose to the brain during periods of starvation. A person's brain normally consumes about 120 grams of glucose per day. Because fat is a poor source of glucose, the prime source from which glucose can be made after glycogen stores (sufficient for a day or two of starvation) have been depleted is muscle protein. Muscle is about 80% water and about 20% protein. At maximum efficiency, during a period of complete starvation, the adult converts about one kilogram of muscle per day for the 120 gram glucose requirement. In addition to skeletal muscle, the heart also suffers loss of its muscle mass. Since the major physiologic functions of living such as respiration, heart action, and locomotion are powered by muscle, severe muscle loss contributes to rapid death.

### SUMMARY OF THE INVENTION

None of the previous uses of creatine exploit its proposed key role in protein synthesis as a way of treating muscle and weight loss, especially loss associated with liver and kidney disease, nor has creatine been recommended for dialysis or other parenteral use. The present invention is based on the realization by the inventor that weight loss associated with liver and kidney diseases is a result of a failure to produce adequate amounts of creatine. In accordance with this realization, the present invention provides a novel method of treating patients for muscle loss. Accordingly, it is an object of the present invention to counteract the muscle loss associated with liver disease and kidney disease by supplying effective amounts of creatine.

The present invention provides a method of treating muscle loss in a subject suffering muscle loss from reduced production of endogenous creatine. The method is practiced by administering an effective amount of a creatine compound, which can be creatine, a pharmaceutically acceptable creatine precursor, analog, or pro-drug, and biologically active salts thereof. For patients undergoing kidney or liver dialysis therapy, the creatine compound can be administered in solution by mouth or as a regular component of dialysis fluid. The method of this invention can be extended to treat patients with low food intake, parenterally fed patients and “failure to thrive” infants, and could be practiced by administering creatine in a peritoneal dialysis solution.

The present invention also provides a dialysis fluid for treating muscle loss, comprising a creatine compound. Preferably, the dialysis fluid contains creatine monohydrate and is used to treat liver and kidney patients.

### DETAILED DESCRIPTION OF THE INVENTION

As used herein, “creatine compound” refers to creatine, pharmaceutically acceptable creatine analogs, precursors, and pro-drugs which metabolize to creatine, and biologically active salts thereof. In particular, a creatine compound can be creatine monohydrate, creatine phosphate, the creatine analog cyclocreatine, the creatine precursor guanidoacetic acid, and hydrosoluble organic salts of creatine as described in U.S. Pat. No. 5,973,199 of Negrisoli et al., which is hereby incorporated by reference. Preferably, the creatine compound is creatine monohydrate.

The term “treating” refers to all types of control such as prophylaxis, cure, relief of symptoms, attenuation of symptoms and arrest of advance. In particular, treating refers to counteracting muscle loss associated with liver, kidney and other diseases and conditions.

The present inventor has realized that weight loss associated with kidney and liver diseases is a consequence of a failure to synthesize creatine. The present inventor has further realized that a diseased kidney or liver cannot produce normal levels of creatine. Without creatine, kidney and liver patients cannot synthesize muscle protein. Any amino acids liberated by breakdown of protein are consumed for synthesis of glucose and energy rather than for building muscle. Thus, patients with liver and kidney disease lose weight. The present inventor believes this is the first reported proposal of a connection between weight loss accompanying liver and kidney diseases and failure to synthesize sufficient creatine.

The danger of muscle loss is well known, and attempts are made to feed a high protein intake to patients with liver or kidney disease. However, liver patients have difficulty disposing of the toxic ammonia produced by metabolism of amino acids since a damaged liver cannot dispose of ammonia as easily as it can non-toxic urea. Thus, patients with chronic liver disease have difficulty adhering to high protein diets. Further, the present inventor believes that a high protein diet alone is an inadequate therapy for liver and kidney patients so long as such patients lack adequate levels of creatine.

Both the liver and kidney are normally required not only to produce necessary creatine, but also to detoxify and excrete toxic by-products of the consumption of amino acids. Loss of muscle and death frequently results from disease of either organ, due to the cachexia of severe muscle loss and the toxicity of the by-products (e.g. ammonia) of excessive use of body protein for brain glucose. Renal dialysis and peritoneal dialysis are used regularly in renal disease to remove the toxic breakdown products, and creatine can be included as a regular component of dialysis fluids.

Creatine has been used clinically as a nutritional supplement to improve the strength, speed and size of athletes' muscles. However, the key positive role proposed for creatine phosphate in muscle protein synthesis has been overlooked by all who have studied or discussed the use of creatine in supporting energy enhancement, particularly in the field of athletics. Even though possible growth of muscle has been observed in some cases, it has been discounted as the collection of water due to the osmotic effect of creatine accumulation in the muscle tissue, rather than actual tissue protein growth. This conclusion cannot be supported by osmotic calculations based on the minimal information available, but it is a general assumption. The failure of the athletic use of creatine to reveal a direct effect of creatine phosphate on muscle protein synthesis is likely due to the use of creatine in well-muscled athletes whose relative increase in creatine-stimulated muscle mass would go unnoticed.

In accordance with this invention, a patient suffering muscle loss from reduced production of endogenous creatine is administered a creatine compound to treat the muscle loss. The creatine compound can be administered through routes well known in the art such as oral, intravenous, or dialysis. When provided orally, the creatine compound can be in the form of a pill, tablet, capsule, powder, solution, suspension and the like. Whatever the route, the compound can be mixed with additional components such as buffers, salts, adjuvants, solubilizers, carriers, flavoring agents, sugars, minerals, and vitamins.

In some cases, adequate levels of creatine cannot be attained orally. In such cases, creatine can be administered by dialysis to achieve higher levels. Either hemodialysis or peritoneal dialysis can be performed. In hemodialysis, the patient's blood is passed through an artificial kidney having a membrane that acts to clean the blood. In peritoneal dialysis, a dialysis solution is introduced into the patient's peritoneal cavity where the peritoneum can act as a semi-permeable membrane for exchanging solutes between the dialysis solution and the patient's blood.

Another advantage of creatine administration by dialysis is the higher blood levels of creatine achieved during dialysis compare with oral administration. Higher blood levels could result in rapidly rising intracellular concentrations of creatine. A further advantage of dialysis is that undesirable guanidine analogs or creatine precursors can be dialyzed out during dialysis, reducing their levels in comparison with heavy oral creatine therapy.

In a preferred embodiment, creatine in the form of creatine monohydrate can be added to a dialysis solution at concentrations up to about 1.5 grams/100 ml of solution, the solubility limit of creatine monohydrate in aqueous solutions. Preferably, the concentration of creatine monohydrate is about 1.5 grams/100 ml of solution, or the maximum solubility attainable in a particular dialysis solution, which depends in part upon the other components of the solution. As is readily understood by those working in the field, a creatine fortified dialysis solution can include other solutes such as sodium, potassium, glucose, bicarbonate, magnesium, calcium and chloride. The concentrations of these other solutes can be adjusted to assure proper plasma levels in the patient.

Creatine administration, and particularly creatine fortified dialysis, can be beneficial for counteracting muscle loss in patients suffering from kidney and liver diseases. Many kidney patients regularly undergo dialysis, and creatine can be added as a regular component of dialysis fluids. For patients with severe liver disease, creatine fortified dialysis may be a way of preserving failing physiology until transplant.

Other types of muscle loss that can be amenable to the beneficial effects of creatine administration, and particularly parenteral administration, are anorexia nervosa, chronic gastrointestinal disease, and severe wounds that interfere with oral intake of food. There is a large group of patients who must be fed parenterally and who may benefit by obviating the need for complete dependence on endogenous creatine synthesis. Creatine may also be of value in the chronic parenteral feeding of vegetarians with mild liver or kidney ailments or other causes of severe weight loss.

“Failure to thrive” defines infants who delay for months before a normal growth rate takes place. These children may also benefit from creatine administration.

Although the subjects described herein are human subjects, the invention can be extended to animal subjects with diseases or conditions associated with muscle loss.

An effective amount of a creatine compound is any amount that achieves the goal of therapy. For example, an effective amount for prophylaxis is any amount necessary to maintain muscle mass. Alternatively, an effective amount for counteracting muscle loss is any amount that leads to increased muscle mass. As would be apparent to those working in the field, an effective amount in any given case depends upon the particular formulation employed, the route of administration, the site and rate of administration, the clinical tolerance of the patient involved, the age and health of the patient, the pathological condition afflicting the patient and the like. The patient's condition can be monitored and the dosages varied accordingly.

To measure muscle mass, excretion of the compound creatinine can be monitored. Creatinine, which is formed from creatine by irreversible loss of a molecule of water, has no known function. It is excreted by the normal human in almost exact proportion to the muscle mass of the individual. Its daily excretion is equivalent to about 2 grams of creatine. As would be expected, the daily excretion of creatinine by the female, also proportional to muscle mass, is smaller than the male. It has been suggested that creatinine is formed by muscle contraction, but the number of molecules of creatinine excreted represents only a small fraction of the molecules of creatine phosphorylated and dephosphorylated per day. No enzymatic process has been found for the formation of creatinine.

About 100 grams of creatine are present in the normal body, and if all of the creatine were converted to creatinine, about 57 grams would form. If synthesis of creatine stops, muscle loss occurs and the excretion of creatinine diminishes proportionately. Although details concerning creatinine function and synthesis remain to be determined, creatinine has been found to be a good measure of muscle mass.

As currently envisioned, the need for, and effectiveness of, creatine treatment can be determined by measuring the twenty-four hour urinary output of creatinine, a standard laboratory test. This measurement will give a baseline value for initiating creatine administration. A normal male excretes about 1.50 grams of creatinine per day. If excretion of creatinine is below the normal amount relative to a patient's weight, supplemental creatine can be administered until the excretion of creatinine is approximately normal. Increase in muscle mass would be shown by increase in creatinine excretion.

Other ways of monitoring changes in muscle mass include measuring ⁴⁰K, a natural isotope of potassium that is present primarily in muscle tissue and that requires a special counting apparatus, and examination by magnetic resonance imaging, which can give additional information on localization and density of muscle tissue.

### REFERENCES

1. Bessman, S. P. and Fonyo, A. The possible role of the mitochondria bound creatine kinase in regulation of mitochondrial respiration. Biochem. Biophys. Res. Comm. 22, 597-602 (1966).

2. Bessman, S. P. Hexokinase—Acceptor theory of insulin action. New evidence. Israel J. Med. Sci. 8, 344 (1972).

3. Carpenter, C. L., Mohan, C. and Bessman, S. P. Inhibition of protein and lipid synthesis in muscle by 2,4-dinitrofluorobenzene, an inhibitor of creatine phosphokinase. Biochem. Biophys. Res. Comm. 111, 884-889 (1983).

4. Savabi, F., Carpenter, C. L., Mohan, C. and Bessman, S. P. The polysome as a terminal for the creatine phosphate energy shuttle. Biochem. Med. Metab. Biol. 40, 291-298 (1988).

5. Ingwall, J. S., Morales, M. F. and Stockdale, F. E. Creatine and the control of myosin synthesis in differentiating skeletal muscle. Proc. Natl. Acad. Sci. USA. 69, 2250- 2253 (1972).

6. Stockler, S., Hanefeld, F. and Frahm, J. Creatine replacement therapy in guanidinoacetate methyltransferase deficiency, a novel inborn error of metabolism. Lancet 348, 789-790 (1996 ).

7. Stockier, S., Marescau, B., De Deyn, P. P., Trijbels, J. M. F. and Hanefeld, F. Guanidino compounds in guanidinoacetate methyltransferase deficiency, a new inborn error of creatine synthesis. Metabolism 46, 1189-1193 (1997).

1. A method of treating muscle loss in a subject suffering muscle loss from reduced production of endogenous creatine, comprising administering an effective amount of a creatine compound.

2. The method of claim 1 in which the subject is
a subject with kidney disease,
a subject with liver disease,
a subject suffering from anorexia nervosa,
a subject with chronic gastrointestinal disease,
a subject with severe wounds that interfere with oral intake of food,
a parenterally fed subject, or
a failure-to-thrive infant.

3. The method of claim 2 in which the subject is a subject with kidney disease or a subject with liver disease.

4. The method of claim 2 in which the subject is a vegetarian with mild kidney disease.

5. The method of claim 2 in which the subject is a vegetarian with mild liver disease.

6. The method of claim 1 in which the creatine compound is creatine, a pharmaceutically acceptable creatine analog, a pharmaceutically acceptable creatine precursor, a pharmaceutically acceptable creatine pro-drug, or biologically active salts thereof.

7. The method of claim 1 in which the creatine compound is creatine monohydrate, creatine phosphate, cyclocreatine, guanidoacetic acid, or hydrosoluble organic salts of creatine.

8. The method of claim 7 in which the creatine compound is creatine monohydrate.

9. The method of claim 1 in which the creatine compound is administered by dialysis.

10. The method of claim 9 in which the creatine compound is creatine monohydrate.

11. The method of claim 10 in which creatine monohydrate has a concentration of up to about 1.5 g/100 ml of dialysis fluid.

12. The method of claim 11 in which creatine monohydrate has a concentration of about 1.5 g/100 ml of dialysis fluid.

13. A method of counteracting muscle loss associated with diseases of liver and kidney, comprising administering an effective amount of creatine monohydrate to a subject suffering such muscle loss, the creatine monohydrate administered by dialyzing the subject with a dialysis fluid containing creatine monohydrate.

14. A dialysis fluid for treating muscle loss, the dialysis fluid containing a creatine compound.

15. The dialysis fluid of claim 14 in which the creatine compound is creatine, a pharmaceutically acceptable creatine analog, a pharmaceutically acceptable creatine precursor, a pharmaceutically acceptable creatine pro-drug, or biologically active salts thereof.

16. The dialysis fluid of claim 14 in which the creatine compound is creatine monohydrate, creatine phosphate, cyclocreatine, guanidoacetic acid, or hydrosoluble organic salts of creatine.

17. The dialysis fluid of claim 16 in which the creatine compound is creatine monohydrate.

18. The dialysis fluid of claim 17 in which creatine monohydrate has a concentration of up to about 1.5 g/100 ml of fluid.

19. The dialysis fluid of claim 18 in which creatine monohydrate has a concentration of about 1.5 g/100 ml of fluid.

20. The dialysis fluid of claim 14 in which the dialysis fluid is a hemodialysis fluid.

21. The dialysis fluid of claim 14 in which the dialysis fluid is a peritoneal dialysis fluid.

22. A dialysis fluid for treating muscle loss associated with diseases of liver and kidney, the dialysis fluid containing creatine monohydrate.
